(19) United States
(12) Patent Application Publication
(10) Pub. No.: US 2026/0294239 A1
(43) Pub. Date: Oct. 1, 2026

(54) OPHTHALMIC IMAGING SYSTEM

(71) Applicant: Young Optics Inc., Hsinchu Science Park (TW)

(72) Inventors: CHING-LUN LIN, Hsinchu Science Park (TW); CHING-SHENG CHANG, Hsinchu Science Park (TW); CHENG-HUNG CHEN, Hsinchu Science Park (TW)

(21) Appl. No.: 19/702,062

(22) Filed: Jun. 9, 2026

Related U.S. Application Data
(63) Continuation-in-part of application No. 18/621,857, filed on Mar. 29, 2024.

(30) Foreign Application Priority Data
May 18, 2023 (TW) 112118547
Feb. 5, 2024 (TW) 113104518

(51) Int. Cl. A61B 3/12; A61B 3/00

(52) U.S. Cl. CPC A61B 3/12; A61B 3/0008; A61B 3/0091

(57) ABSTRACT

An ophthalmic imaging system includes a first lens group, an illumination light source, an imaging module, a fixation light, a reflective element, at least one light-blocking member, and a first polarizer. The first lens group includes an aspheric first lens. The fixation light provides a light beam and includes at least three light-emitting components. The reflective element is configured to reflect the light beam and direct the light beam towards the aspheric first lens. The light-blocking member is disposed downstream from and in a light path of the fixation light and disposed upstream from and in a light path of the reflective element. The first polarizer is disposed downstream from and in the light path of the fixation light and disposed upstream from and in the light path of the reflective element to polarize the light beam.

### CROSS-REFERENCE TO RELATED APPLICATION

The present application is a continuation-in-part of U.S. patent application Ser. No. 18/621,857, filed Mar. 29, 2024, which claims the priority benefit of Taiwan application serial no. 112118547, filed May 18, 2023, and Taiwan application serial no. 113104518, filed Feb. 5, 2024. The entirety of the above-mentioned patent applications is hereby incorporated by reference herein and made a part of this specification.

### BACKGROUND

### FIELD OF THE INVENTION

The invention relates to an imaging system, more particularly to an ophthalmic imaging system.

### Description of the Related Art

Typically, fundus photography requires an illumination light source powerful enough to illuminate the fundus. The illumination light sources traditionally used for fundus photography are often placed at a position far from the eyepiece (the lens closest to the eyeball), resulting in poor illumination efficiency. Therefore, the integration of extra optical components is required to improve the illumination efficiency.

### BRIEF SUMMARY OF THE INVENTION

In order to achieve one or a portion of or all of the objects or other objects, one embodiment of the invention provides an ophthalmic imaging system including a first lens group having a positive refractive power, an illumination light source, an imaging module, a fixation light, a reflective element, at least one light-blocking member, and a first polarizer. The first lens group includes an aspheric first lens, and the illumination light source is capable of forming an illumination zone between the illumination light source and the first lens group. The imaging module is capable of forming an imaging zone between the imaging module and the first lens group, and the imaging zone covers an optical axis of the first lens group. The fixation light provides a light beam and includes at least three light-emitting components. The reflective element is disposed downstream from and in a light path of the fixation light and disposed upstream from and in a light path of the aspheric first lens. The light-blocking member is disposed downstream from and in the light path of the fixation light and disposed upstream from and in a light path of the reflective element. The first polarizer is disposed downstream from and in the light path of the fixation light and disposed upstream from and in the light path of the reflective element to polarize the light beam.

Another embodiment of the invention provides an ophthalmic imaging system including a first lens group having a positive refractive power, an illumination light source, an imaging module, a fixation light, a reflective element, at least one light-blocking member, and a first polarizer. The first lens group includes an aspheric first lens, and the imaging module is disposed on an optical axis of the first lens group. The fixation light provides a light beam and includes at least three light-emitting components. The reflective element is configured to reflect the light beam and direct the light beam towards the aspheric first lens. The light-blocking member is disposed downstream from and in a light path of the fixation light and disposed upstream from and in a light path of the reflective element. The first polarizer is disposed downstream from and in the light path of the fixation light and disposed upstream from and in the light path of the reflective element to polarize the light beam.

According to the above embodiments, by setting the positional relationships among the illumination light source, the first lens group, the fixation light, the reflective element and the fold mirrors, the components of the entire system can be arranged more compactly within a limited barrel space to facilitate the miniaturization of an ophthalmic imaging system. Moreover, by placing the first and second light-blocking members in a light path between the fixation light and the reflective element, the position and angle of the light beams emitted by the fixation light can be restricted to reduce aberrations in the image beams projected onto the fundus. Furthermore, a first polarizer and a second polarizer are provided to polarize the light beam emitted by the fixation light before entering the reflective element and to polarize the light beam reflected by the fundus towards the imaging module. The polarization directions of the first polarizer and the second polarizer are different to prevent light beams emitted by the illumination light source or the fixation light from directly entering the imaging module, thus reducing stray light and improving the imaging quality of the ophthalmic imaging system.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 shows a schematic diagram of an ophthalmic imaging system according to an embodiment of the invention.

FIG. 2 shows a schematic diagram of an ophthalmic imaging system according to another embodiment of the invention.

FIG. 3 shows a schematic diagram of an ophthalmic imaging system according to another embodiment of the invention.

FIG. 4A and FIG. 4B are schematic diagrams respectively showing an illumination light path and an imaging light path of the ophthalmic imaging system of FIG. 3.

FIG. 5 is a schematic diagram of an ophthalmic imaging system according to another embodiment of the invention.

FIG. 6A is a schematic diagram of an ophthalmic imaging system according to another embodiment of the invention, and FIG. 6B is a schematic diagram showing an arrangement of multiple light-emitting components of FIG. 6A.

FIG. 7A is a schematic diagram of an ophthalmic imaging system according to another embodiment of the invention, and FIG. 7B is a schematic diagram showing an arrangement of multiple light-emitting components of FIG. 7A.

### DETAILED DESCRIPTION OF THE INVENTION

In the following detailed description of the preferred embodiments, directional terminology, such as “top,” “bottom,” “front,” “back,” etc., is used with reference to the orientation of the Figure(s) being described. The components of the invention can be positioned in a number of different orientations. As such, the directional terminology is used for purposes of illustration and is in no way limiting. Further, “First,” “Second,” etc., as used herein, are used as labels for nouns that they precede, and do not imply any type of ordering (e.g., spatial, temporal, logical, etc.).

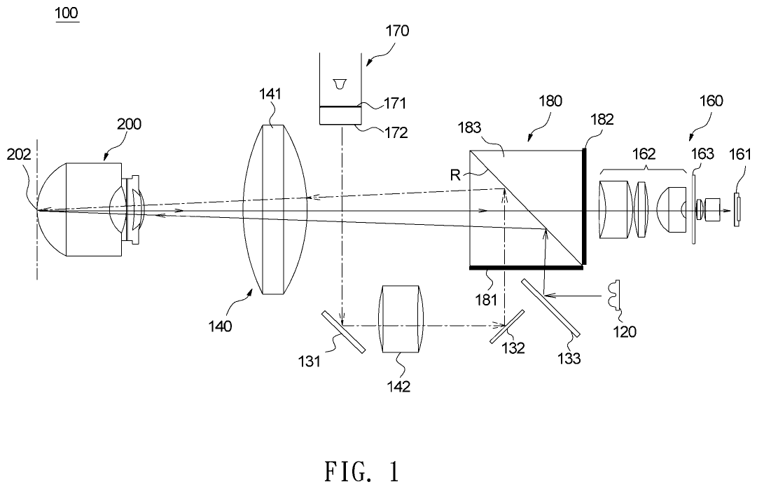

FIG. 1 shows a schematic diagram of an ophthalmic imaging system according to an embodiment of the invention. As shown in FIG. 1, the ophthalmic imaging system 100 includes an illumination light source 120, a first lens group (eyepiece group) 140, an imaging module 160, a fixation light 170, a reflective element 180, a first light-blocking member 171 and a second light-blocking member 172. Specifically, the illumination light source 120 may include an infrared light source and a white light source, and the infrared or white light source can be, for example, a light-emitting diode (LED), an organic light-emitting diode (OLED), or a laser diode (LD). The first lens group 140 has a positive refractive power and at least includes an aspheric lens 141, and the aspheric lens 141 may be a molded-glass lens. The illumination light source 120 is capable of forming an illumination zone between the illumination light source 120 and the first lens group 140, the imaging module 160 is capable of forming an imaging zone between the imaging module 160 and the first lens group 140, and the imaging zone covers an optical axis of the first lens group 140. In this embodiment, the molded-glass aspheric lens 141 is the closest lens to the object side (closest to the eyeball 200) within the first lens group 140 and has a non-zero refractive power. Specifically, the reflective element 180 may be a polarizing beam splitter (PBS) prism 183 having a reflective surface R. The reflective surface R may direct light beams traveling through the imaging and illumination paths towards the first lens group 140, and then the light beams may pass through the first lens group 14 to reach the fundus (retina) 202 of an individual being examined (a subject), thereby enabling illumination and image pick-up functions of the ophthalmic imaging system 100. In one embodiment, the first light-blocking member 171 and the second light-blocking member 172 are aperture stops, the fixation light 170 may have at least three light-emitting components, such as three light-emitting diodes (LEDs), and the three light-emitting components may each produce its own light and collectively provide a light beam. The fixation light 170 can keep the subject's eye fixed at a specific observation angle to prevent involuntary eye movements. The three differently positioned light-emitting components such as LEDs can fix the subject's eye at a certain angle based on the particular requirements. Moreover, the light-blocking members 171 and 172 can be disposed downstream from and in a light path of the fixation light 170 and disposed upstream from and in a light path of the reflective element 180. Such arrangement is allowed to restrict the position and angle of the light beam emitted by the fixation light 170 to reduce aberrations in the image beams of the fixation light 170 projected onto the fundus.

In this embodiment, the ophthalmic imaging system 100 also includes a first fold mirror 131, a second fold mirror 132, a third fold mirror 133, a relay lens 142, a first polarizer 181, and a second polarizer 182. The first fold mirror 131 is disposed downstream from and in a light path of the fixation light 170 and disposed upstream from and in a light path of the reflective element 180 to direct the light beam emitted by the fixation light 170 towards the reflective element 180. The second fold mirror 132 is disposed downstream from and in a light path of the fixation light 170 and the first fold mirror 131 and disposed upstream from and in the light path of the reflective element 180. The relay lens 142 is disposed in a light path between the first fold mirror 131 and the second fold mirror 132. The third fold mirror 133 is disposed downstream from and in a light path of the illumination light source 120 and disposed upstream from and in a light path of the reflective element 180. The first polarizer 181 is disposed downstream from and in a light path of the fixation light 170, the first fold mirror 131 and the second fold mirror 132. The second polarizer 182 is disposed downstream from and in a light path of the reflective element 180 and disposed upstream from and in a light path of the imaging module 160 to polarize the light beam from the illumination light source 120. Specifically, a light beam emitted from the fixation light 170 is guided by the first fold mirror 131, the relay lens 142 and the second fold mirror 132 towards the reflective element 180, and then the light beam is deflected by the reflective element 180 to pass through the first lens group 140 to form an image on the fundus 202 of the subject. Furthermore, the infrared and/or white light beam emitted by the illumination light source 120 is reflected by the third fold mirror 133 towards the reflective element 180, and then reflected by the reflective element 180 to pass through the first lens group 14 to illuminate the fundus 202 of the subject. The fold mirrors 131-133 are disposed to fold the light paths; therefore, the fixation light 170, the relay lens 142 and the reflective element 180 do not need to be arranged along a single straight optical axis. Such configuration allows the fixation light path to be accommodated within a limited barrel space, thereby making the overall optical layout more compact. Moreover, the relay lens 142 is configured to relay the divergent light beam emitted from the fixation light 170, so that the divergent light beam can further propagate along the downstream portion of the fixation light path toward a farther position.

In this embodiment, because the first polarizer 181 is disposed downstream of the fixation light 170 and upstream of the reflective element 180 along the light path, the light beam from the fixation light 170 is polarized prior to reaching the reflective element 180. This arrangement may reduce stray light within the imaging zone, thereby enhancing the image quality. Similarly, the second polarizer 182 is disposed downstream of the reflective element 180 and upstream of the imaging module 160 along the light path to polarize the light beam that is reflected by the fundus 202 of the eyeball 200 towards the imaging module 160. The polarization directions of the first polarizer 181 and the second polarizer 182 are different to prevent light beams emitted by the illumination light source 120 or the fixation light 170 from directly entering the imaging module 160 through the reflective element 180, with these undesired light beams entering the imaging module 160 contributing to stray light, and therefore the image quality of the ophthalmic imaging system 100 can be improved.

In this embodiment, the imaging module 160 includes an image sensor 161 and a second lens group 162. The second lens group 162 may include multiple lenses with refractive powers or a singlet lens with a refractive power. The imaging module 160 may further include an infrared light filter 163 disposed in a light path between the image sensor 161 and the second lens group 162. The fundus 202 reflects the illumination light beam to form an image beam, and the image beam sequentially passes through the first lens group (eyepiece group) 140 and the second lens group 162 and is then focused onto the image sensor 161 of the imaging module 160, thereby forming a fundus image. The ophthalmic imaging system 100 may further include a display (not shown) to display the fundus photograph output by the image sensor 161.

Based on the design of various embodiments of the invention, by setting the positional relationships among the illumination light source, the first lens group, the fixation light, the reflective element and the fold mirrors, the components of the entire system can be arranged more compactly within a limited barrel space to facilitate the miniaturization of an ophthalmic imaging system. Moreover, by placing the first and second light-blocking members in a light path between the fixation light and the reflective element, the position and angle of the light beams emitted by the fixation light can be restricted to reduce aberrations in the image beams projected onto the fundus. Furthermore, a first polarizer and a second polarizer are provided to polarize the light beam emitted by the fixation light before entering the reflective element and to polarize the light beam reflected by the fundus towards the imaging module. The polarization directions of the first polarizer and the second polarizer are different to prevent light beams emitted by the illumination light source or the fixation light from directly entering the imaging module, thus reducing stray light and improving the imaging quality of the ophthalmic imaging system.

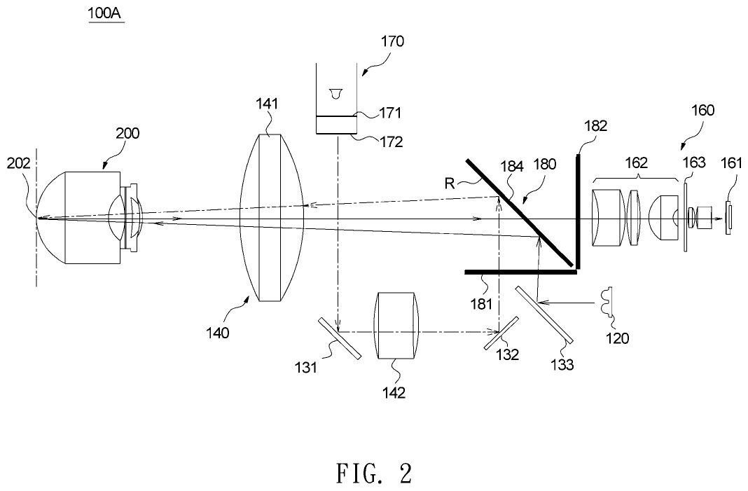

FIG. 2 is a schematic diagram of an ophthalmic imaging system according to another embodiment of the invention. As shown in FIG. 2, the reflective element 180 of the ophthalmic imaging system 100A is implemented as a plate-type polarizing beam splitter, such as a PBS plate 184, instead of a prism-type polarizing beam splitter. Since the PBS plate 184 occupies less space than the prism-type polarizing beam splitter, the illumination light source 120, the fixation light 170 and the imaging module 160 can be arranged more flexibly around the PBS plate 184, thereby facilitating miniaturization of the ophthalmic imaging system 100A.

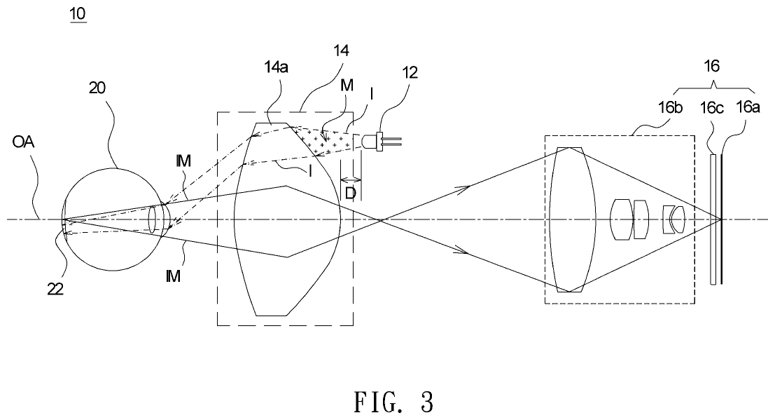

FIG. 3 is a schematic diagram of an ophthalmic imaging system according to another embodiment of the invention. As shown in FIG. 3, the ophthalmic imaging system 10 includes an illumination light source 12, a first lens group (eyepiece group) 14, and an imaging module 16. The illumination light source 12 may include at least one light-emitting component, such as a light-emitting diode (LED), an organic light-emitting diode (OLED), or a laser diode (LD). The eyepiece group 14 has a positive refractive power and may include an aspheric lens 14a. In this embodiment, the aspheric lens 14a is the lens within the eyepiece group 14 that is closest to an object side of the eyepiece group 14 (i.e., closest to the eyeball 20) and has a non-zero refractive power. The illumination light source 12 is capable of forming an illumination optical zone M between the illumination light source 12 and the eyepiece group 14, and the illumination optical zone M does not overlap or contact an optical axis OA of the eyepiece group 14; that is, the illumination zone formed by the illumination light source 12 does not include the optical axis OA of the eyepiece group 14. One side of the eyepiece group 14 may face the eyeball 20, and an illumination light beam I generated by the illumination light source 12 may be refracted by the eyepiece group 14 having a positive refractive power toward the fundus 22 of the eyeball 20. In this embodiment, a light-emitting position of the illumination light source 12 is offset from the optical axis OA of the eyepiece group 14, and the illumination light beam I generated by the illumination light source 12 is directly incident on the eyepiece group 14, so components such as a relay lens may be omitted. In this embodiment, a positional relationship between the illumination light source 12 and the eyepiece group 14 may satisfy a condition of D<N×0.9, where N is the reciprocal of the refractive power of the eyepiece group 14, and D is a distance between the illumination light source 12 and the eyepiece group 14 in a direction parallel to the optical axis OA of the eyepiece group 14. Satisfying the above condition may bring the illumination light source 12 closer to the eyepiece group 14 and place the illumination light source 12 within the focal length range of the eyepiece group 14, thereby reducing loss of large-angle rays during transmission of the illumination light and reducing ineffective light that cannot reach the fundus, so as to prevent the ineffective light from being transmitted in the lens imaging region to cause ghost images or generate stray light. In one embodiment, the reciprocal of the refractive power of the eyepiece group 14, i.e., the focal length value, may range from 15 mm to 30 mm, and the distance from the illumination light source 12 to the eyepiece group 14 in the direction of the optical axis OA of the eyepiece group 14 may range from 13 mm to 27 mm.

In this embodiment, the eyepiece group 14 may be a single aspheric lens, but the invention is not limited thereto. The imaging module 16 is disposed on the optical axis OA of the eyepiece group 14 and is located on one side of the eyepiece group 14 opposite to the eyeball 20. In this embodiment, the imaging module 16 includes an image sensor 16a and a lens group 16b, and the imaging module 16 may further include an infrared light filter 16c disposed between the image sensor 16a and the lens group 16b. The fundus 22 reflects the illumination light beam I to form an image beam IM, and the image beam IM sequentially passes through the eyepiece group 14 and the lens group 16b and is then focused onto the image sensor 16a of the imaging module 16, thereby forming a fundus image. The light paths of all image beams IM that are emitted from the fundus 22 and finally form an image on the image sensor 16a together constitute an imaging optical zone between the imaging module 16 and the eyepiece group 14; in other words, the imaging module 16 cannot receive image light from outside the imaging optical zone. The ophthalmic imaging system 10 may further include a display (not shown) to display the fundus image output by the image sensor 16a.

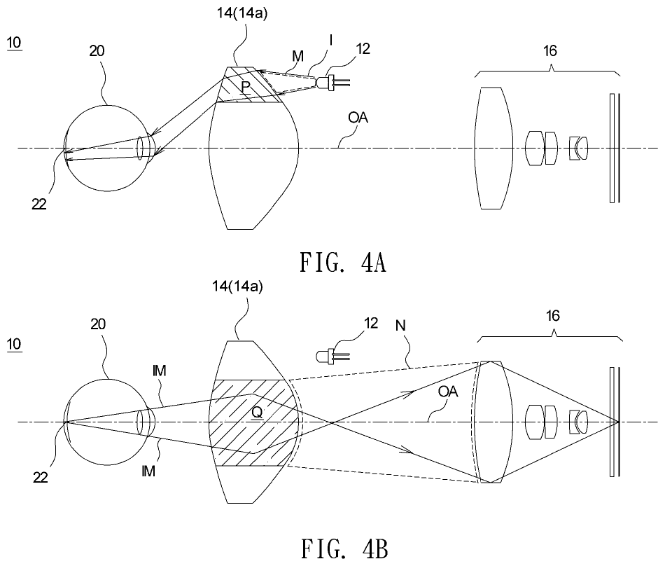

FIGS. 4A and 4B are schematic diagrams respectively showing an illumination light path and an imaging light path of the ophthalmic imaging system of FIG. 3. As shown in FIG. 4A, light emitted by the illumination light source 12 substantially only passes through a peripheral optical region P of the eyepiece group 14, and the peripheral optical region P may provide a light-converging effect for the illumination light beam I emitted by the illumination light source 12, so as to refract the illumination light beam I toward the fundus 22. That is, the illumination optical zone M formed between the illumination light source 12 and the eyepiece group 14 corresponds to the peripheral optical region P of the eyepiece group 14. Moreover, as shown in FIG. 4B, all light reflected from the fundus 22 is transmitted through the eyepiece group 14 to the imaging module 16 for imaging. Therefore, the light paths of all image beams IM that are emitted from the fundus 22 and finally form an image on the image sensor 16a constitute, between the imaging module 16 and the eyepiece group 14, an imaging optical zone N that includes the optical axis OA of the eyepiece group 14 and is arranged with the optical axis OA of the eyepiece group 14 as a center. In this embodiment, the imaging module 16 may be disposed in the light path of the eyepiece group 14 and receive light from a central optical region Q of the eyepiece group 14. That is, the imaging optical zone N formed between the imaging module 16 and the eyepiece group 14 corresponds to the central optical region Q of the eyepiece group 14. In this embodiment, the illumination light source 12 is disposed outside the imaging optical zone N, and the peripheral optical region P and the central optical region Q of the eyepiece group 14 do not contact each other. In other words, by means of the design of the embodiments of the invention, the illumination light path and the imaging light path may be substantially separated, thereby preventing ineffective illumination light that does not reach the fundus from entering the imaging optical zone and then being deflected by lenses to cause ghost images or stray light. Accordingly, fundus image quality can be improved, and use of an additional lens for the illumination light source can be eliminated or reduced.

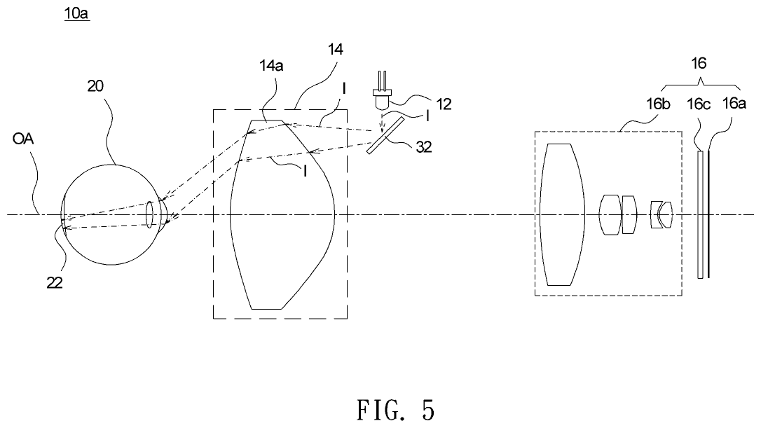

FIG. 5 is a schematic diagram of an ophthalmic imaging system according to another embodiment of the invention. As shown in FIG. 5, a reflecting mirror 32 may be disposed in a light path between the illumination light source 12 and the eyepiece group 14 of the ophthalmic imaging system 10a. The reflecting mirror 32 may be located outside the imaging optical zone and may reflect the illumination light beam I emitted by the illumination light source 12, so that the illumination light beam I is redirected and then incident on the aspheric lens 14a. Generally, the illumination light source 12 needs to be provided with driving electronic components and a fixing mechanism, and the required electronic components or mechanical components may interfere with the imaging light path. With the design of this embodiment, since the reflecting mirror 32 may redirect the illumination light beam I emitted by the illumination light source 12 and thus redirect the illumination light path, arrangement space and arrangement flexibility for imaging optical components can be increased, thereby preventing the electronic components or the mechanical components from interfering with the imaging light path.

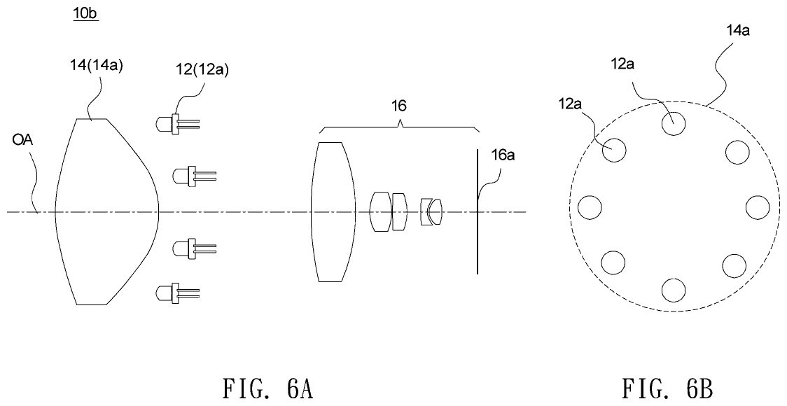

FIG. 6A is a schematic diagram of an ophthalmic imaging system according to another embodiment of the invention, and FIG. 6B is a schematic diagram showing an arrangement of a plurality of light-emitting components of FIG. 6A. As shown in FIGS. 6A and 6B, the illumination light source 12 of the ophthalmic imaging system 10b may include multiple light-emitting components 12a arranged substantially in an annular manner, thereby avoiding insufficient illumination of the fundus that may be caused by using a single light-emitting component. Moreover, in one embodiment, each light-emitting component 12a may be independently turned on and off, and an effect of illuminating different regions of the fundus may be provided by selectively switching on and off the light-emitting components 12a located at different positions.

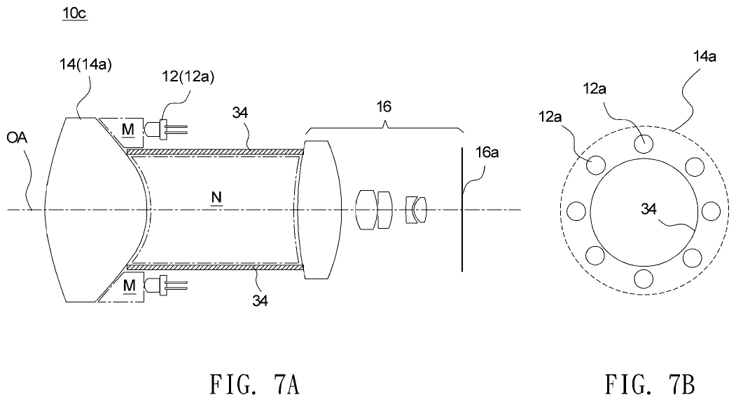

FIG. 7A is a schematic diagram of an ophthalmic imaging system according to another embodiment of the invention, and FIG. 7B is a schematic diagram showing a configuration of the plurality of light-emitting components of FIG. 7A. As shown in FIGS. 7A and 7B, the plurality of light-emitting components 12a are arranged in an annular manner outside the imaging optical zone N, and the ophthalmic imaging system 10c uses a spacer 34, such as an inner lens barrel, to separate the imaging optical zone N from the illumination optical zone M, so as to completely prevent the illumination light from entering the imaging optical zone N and further ensure that ghost images or stray light are not generated. Therefore, according to the design of the embodiments of the invention, even if the imaging optical zone N and the illumination optical zone M slightly overlap, the effect of preventing ghost images or stray light from being generated can still be provided.

Based on the design of various embodiments of the invention, the illumination light source can be brought closer to the lens group and placed within the focal length range of the lens group by setting a positional relationship between the illumination light source and the lens group, thereby reducing loss of large-angle rays during propagation of the illumination light, increasing the amount of illumination light reaching the fundus, and reducing ineffective light that cannot reach the fundus. Moreover, by substantially separating the illumination light path from the imaging light path, ineffective light that does not reach the fundus can also be prevented from entering the imaging optical zone, thereby achieving the effect of preventing ghost images or stray light from being generated and improving fundus image quality.

Though the embodiments of the invention have been presented for purposes of illustration and description, they are not intended to be exhaustive or to limit the invention. Accordingly, many modifications and variations without departing from the spirit of the invention or essential characteristics thereof will be apparent to practitioners skilled in this art. It is intended that the scope of the invention be defined by the claims appended hereto and their equivalents in which all terms are meant in their broadest reasonable sense unless otherwise indicated.

What is claimed is:

1. An ophthalmic imaging system, comprising:
a first lens group having a positive refractive power and comprising an aspheric first lens;
an illumination light source capable of forming an illumination zone between the illumination light source and the first lens group;
an imaging module capable of forming an imaging zone between the imaging module and the first lens group, and the imaging zone covering an optical axis of the first lens group;
a fixation light for providing a light beam, wherein the fixation light comprises at least three light-emitting components;
a reflective element disposed downstream from and in a light path of the fixation light and disposed upstream from and in a light path of the aspheric first lens;
at least one light-blocking member disposed downstream from and in the light path of the fixation light and disposed upstream from and in a light path of the reflective element; and
a first polarizer disposed downstream from and in the light path of the fixation light and disposed upstream from and in the light path of the reflective element to polarize the light beam provided by the fixation light.

2. The ophthalmic imaging system as claimed in claim 1, wherein the aspheric first lens is a glass-molded lens, and the at least one light-blocking member is an aperture stop.

3. The ophthalmic imaging system as claimed in claim 1, wherein the reflective element is a PBS prism or a PBS plate.

4. The ophthalmic imaging system as claimed in claim 1, further comprising a first fold mirror disposed downstream from and in the light path of the fixation light and disposed upstream from and in the light path of the reflective element to reflect and direct the light beam towards the reflective element.

5. The ophthalmic imaging system as claimed in claim 4, further comprising a second fold mirror disposed downstream from and in the light path of the fixation light, downstream from and in a light path of the first fold mirror, and upstream from and in the light path of the reflective element.

6. The ophthalmic imaging system as claimed in claim 5, further comprising a relay lens disposed in a light path between the first fold mirror and the second fold mirror.

7. The ophthalmic imaging system as claimed in claim 5, further comprising a third fold mirror disposed downstream from and in a light path of the illumination light source and disposed upstream from and in a light path of the reflective element.

8. The ophthalmic imaging system as claimed in claim 5, wherein the first polarizer is disposed downstream from and in a light path of the fixation light, the first fold mirror and the second fold mirror.

9. The ophthalmic imaging system as claimed in claim 1, further comprising a second polarizer disposed downstream from and in a light path of the reflective element and disposed upstream from and in a light path of the imaging module to polarize a light beam from the illumination light source.

10. The ophthalmic imaging system as claimed in claim 1, wherein the imaging module includes a second lens group, an infrared light filter and an image sensor, and the infrared light filter is disposed in a light path between the second lens group and the image sensor.

11. An ophthalmic imaging system, comprising:
a first lens group having a positive refractive power and comprising an aspheric first lens;
an illumination light source;
an imaging module disposed on an optical axis of the first lens group;
a fixation light for providing a light beam, wherein the fixation light comprises at least three light-emitting components;
a reflective element configured to reflect the light beam and direct the light beam towards the aspheric first lens;
at least one light-blocking member disposed downstream from and in a light path of the fixation light and disposed upstream from and in a light path of the reflective element; and
a first polarizer disposed downstream from and in the light path of the fixation light and disposed upstream from and in the light path of the reflective element to polarize the light beam provided by the fixation light.

12. The ophthalmic imaging system as claimed in claim 11, wherein the aspheric first lens is a glass-molded lens, and the at least one light-blocking member is an aperture stop.

13. The ophthalmic imaging system as claimed in claim 11, wherein the reflective element is a PBS prism or a PBS plate.

14. The ophthalmic imaging system as claimed in claim 11, further comprising a first fold mirror disposed downstream from and in the light path of the fixation light and disposed upstream from and in the light path of the reflective element to reflect and direct the light beam towards the reflective element.

15. The ophthalmic imaging system as claimed in claim 14, further comprising a second fold mirror disposed downstream from and in the light path of the fixation light, downstream from and in a light path of the first fold mirror, and upstream from and in the light path of the reflective element.

16. The ophthalmic imaging system as claimed in claim 15, further comprising a relay lens disposed in a light path between the first fold mirror and the second fold mirror.

17. The ophthalmic imaging system as claimed in claim 15, further comprising a third fold mirror disposed downstream from and in a light path of the illumination light source and disposed upstream from and in a light path of the reflective element.

18. The ophthalmic imaging system as claimed in claim 15, wherein the first polarizer is disposed downstream from and in a light path of the fixation light, the first fold mirror and the second fold mirror.

19. The ophthalmic imaging system as claimed in claim 11, further comprising a second polarizer disposed downstream from and in a light path of the reflective element and disposed upstream from and in a light path of the imaging module to polarize a light beam from the illumination light source.

20. The ophthalmic imaging system as claimed in claim 11, wherein the imaging module includes a second lens group, an infrared light filter and an image sensor, and the infrared light filter is disposed in a light path between the second lens group and the image sensor.
